(19) United States
(12) Patent Application Publication
(10) Pub. No.: US 2004/0220209 A1
(43) Pub. Date: Nov. 4, 2004

(54) Novel imidazopyridine compounds with therapeutic effect

(76) Inventors: Kosrat Amin, Molndal (SE); Mikael Dahlstrom, Molndal (SE); Peter Nordberg, Molndal (SE); Ingemar Starke, Molndal (SE)

Correspondence Address: WHITE & CASE LLP, PATENT DEPARTMENT, 1155 AVENUE OF THE AMERICAS, NEW YORK, NY, 10036, US

(21) Appl. No.: 10/487,149

(22) PCT Filed: Aug. 21, 2002
(86) PCT No.: PCT/SE02/01489

(30) Foreign Application Priority Data
Aug. 22, 2001 (SE) 0102808-3

(51) Int. Cl. C07D 471/02; A61K 31/4745

(52) U.S. Cl. 514/303; 546/117

(57) ABSTRACT

The present invention relates to imidazo pyridine derivatives of the formula (I) whichinhibit exogenously or endogenously stimulated gastric acid secretion and thus can be usedin the prevention and treatment of gastrointestinal inflammatory diseases. (I)

### TECHNICAL FIELD

The present invention relates to novel compounds, and therapeutically acceptable salts thereof, which inhibit exogenously or endogenously stimulated gastric acid secretion and thus can be used in the prevention and treatment of gastrointestinal inflammatory diseases. In further aspects, the invention relates to compounds of the invention for use in therapy; to processes for preparation of such new compounds; to pharmaceutical compositions containing at least one compound of the invention, or a therapeutically acceptable salt thereof, as active ingredient; and to the use of the active compounds in the manufacture of medicaments for the medical use indicated above.

### BACKGROUND ART

Substituted imidazo[1,2-a]pyridines, useful in the treatment of peptic ulcer diseases, are known in the art, e.g. from EP-B-0033094 and U.S. Pat. No. 4,450,164 (Schering Corporation); from EP-B-0204285 and U.S. Pat. No. 4,725,601 (Fujisawa Pharmaceutical Co.); WO99/55706 and WO99/55705 (AstraZeneca) and from publications by J. J. Kaminski et al. in the Journal of Medical Chemistry (vol.28, 876-892, 1985; vol. 30, 2031-2046, 1987; vol.30, 2047-2051, 1987; vol. 32, 1686-1700, 1989; and vol. 34, 533-541, 1991).

For a review of the pharmacology of the gastric acid pump (the H⁺, K⁺-ATPase), see Sachs et al. (1995) Annu. Rev. Pharmacol. Toxicol. 35: 277-305.

We have now found additional substituted imidazo[1,2-a]pyridines, useful in the treatment of peptic ulcer diseases exhibiting advantageous properties and potency.

### DISCLOSURE OF THE INVENTION

It has surprisingly been found that compounds of the Formula I are particularly effective as inhibitors of the gastrointestinal H⁺, K⁺-ATPase and thereby as inhibitors of gastric acid secretion.

In one aspect, the present invention thus relates to compounds of the general Formula I

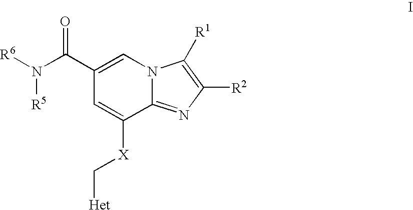

or a pharmaceutically acceptable salt thereof, wherein Het is a 4-, 5-, or 6-membered aromatic or aliphatic heterocyclic group containing at least one nitrogen, oxygen or sulphur atom, substituted with a R³ and a R⁴ group in the ortho positions;

R¹ is

(a) H,

(b) CH₃, or

(c) CH₂OH;

R² is

(a) CH₃, or

(b) CH₂CH₃;

R³ and R⁴ are independently selected from the group of

(a) H,

(b) C₁-C₆ alkyl,

(c) hydroxylated C₁-C₆ alkyl, or

(d) halogen;

R⁵ and R⁶ are independently selected substituents, comprising C, H, N, O, S, Se, P and halogen atoms, which give compounds of Formula I a molecular weight≦600;

R⁵ and R⁶, together with the nitrogen atom to which they are attached, form a saturated or unsaturated ring optionally containing one or more further heteroatoms and;

X is

(a) NH, or

(b) O.

As used herein, the term “C₁-C₆ alkyl” denotes a straight or branched alkyl group having from 1 to 6 carbon atoms. Examples of said C₁-C₆ alkyl includes, but is not limited to, methyl, ethyl, n-propyl, iso-propyl, n-butyl, iso-butyl, sec-butyl, t-butyl and straight- and branched-chain pentyl and hexyl.

The term “halogen” includes fluoro, chloro, bromo and iodo.

The term “4-, 5-, or 6-membered aromatic or aliphatic heterocyclic group containing at least one nitrogen, oxygen or sulphur atom” includes, but is not limited to substituted or unsubstituted azetidine, furan, thiophene, pyrrole, pyrroline, pyrrolidine, dioxolane, oxathiolane, oxazolane, oxazole, thiazole, imidazole, imidazoline, imidazolidine, pyrazole, pyrazoline, pyrazolidine, isoxazole, isothiazole, oxadiazole, furazan, triazole, thiadiazole, pyran, pyridine, piperidine, dioxane, morpholine, dithiane, oxathiane, thiomorpholine, pyridazine, pyrimidine, pyrazine, piperazine, triazine, thiadiazine, dithiazine groups, and shall be understood to include all isomers of the above identified groups. The term “azetidinyl” shall, for example, be understood to include the 2-, and 3-isomers and the terms “pyridyl” and “piperidinyl” shall, for example, by understood to include the 2-, 3-, and 4-isomers.

The pure enantiomers, racemic mixtures and unequal mixtures of two enantiomers are within the scope of the invention. It should be understood that all the diastereomeric forms possible (pure enantiomers, racemic mixtures and unequal mixtures of two enantiomers) are within the scope of the invention. Also included in the invention are derivatives of the compounds of the Formula I that have the biological function of the compounds of the Formula I.

Depending on the process conditions the end products of the Formula I are obtained either in neutral or salt form. Both the free base and the salts of these end products are within the scope of the invention.

Acid addition salts of the new compounds may in a manner known per se be transformed into the free base using basic agents such as alkali or by ion exchange. The free base obtained may also form salts with organic or inorganic acids.

In the preparation of acid addition salts, preferably such acids are used which forms suitably therapeutically acceptable salts. Examples of such acids are hydrohalogen acids such as hydrochloric acid, sulphuric acid, phosphoric acid, nitric acid, aliphatic, alicyclic, aromatic or heterocyclic carboxyl or sulphonic acids, such as formic acid, acetic acid, propionic acid, succinic acid, glycolic acid, lactic acid, malic acid, tartaric acid, citric acid, ascorbic acid, maleic acid, hydroxymaleic acid, pyruvic acid, p-hydroxybensoic acid, embonic acid, methanesulphonic acid, ethanesulphonic acid, hydroxyethanesulphonic acid, halogenbensenesulphonic acid, toluenesulphonic acid or naphthalenesulphonic acid.

Preferred compounds according to the invention are those of the Formula I or a pharmaceutically acceptable salt thereof, wherein Het is

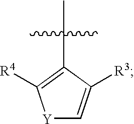

R¹ is

(a) H,

(b) CH₃, or

(c) CH₂OH;

R² is

(a) CH₃, or

(b) CH₂CH₃;

R³ and R⁴ are independently selected from the group of

(a) H,

(b) C₁-C₆ alkyl,

(c) hydroxylated C₁-C₆ alkyl, or

(d) halogen;

R⁵ and R⁶ are independently selected substituents, comprising C, H, N, O, S, Se, P and halogen atoms, which give compounds of Formula I a molecular weight≦600;

R⁵ and R⁶, together with the nitrogen atom to which they are attached, form a saturated or unsaturated ring optionally containing one or more further heteroatoms;

X is

(a) NH, or

(b) O; and

Y is S, SO, SO₂, O, NH, C═N, and N═C.

More preferred compounds according to the invention are those of the Formula I or a pharmaceutically acceptable salt thereof. wherein Het is

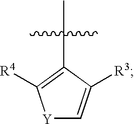

R¹ is CH₃ or CH₂OH;

R² is CH₃, or CH₂CH₃;

R³ and R⁴ are independently selected from the group of H, C₁-C₆ alkyl, hydroxylated C₁-C₆ alkyl, and halogen;

R⁵ and R⁶ are independently

(a) H,

(b) C₁-C₆ alkyl,

(c) mono or dihydroxylated C₁-C₆ alkyl,

(d) C₁-C₆ alkoxy-substituted C₁-C₆ alkyl,

(e) C₂-C₆ alkenyl,

(f) C₂-C₆ alkynyl,

(g) halogenated C₁-C₆ alkyl,

(h) C₃-C₈ cycloalkyl,

(i) cycloalkyl-substituted C₁-C₆ alkyl,

(j) aryl, in which aryl represents phenyl, pyridyl, thienyl or furanyl, optionally substituted by one or more substituents selected from halogen, C₁-C₆ alkyl, C₁-C₆ alkoxy, CF₃, OH, nitro, amino, C₁-C₆ alkyl-NH—, (C₁-C₆ alkyl)₂-N—, or CN,

(k) aryl substituted C₁-C₆ alkyl, in which aryl represents phenyl, pyridyl, thienyl or furanyl, optionally substituted with one or more substituents selected from halogen, C₁-C₆ alkyl, C₁-C₆ alkoxy, CF₃, OH, nitro, amino C₁-C₆ alkyl-NH—, (C₁-C₆ alkyl)₂-N—, or CN,

(l) R⁸-alkyl, in which R⁸ is NH₂C═O—, C₁-C₆ alkyl-NHC═O—, (C₁-C₆ alkyl)₂NC═O—, C₁-C₆ alkyl-OOC—, NH₂SO₂—, C₁-C₆ alkyl-SO₂NH—, ArSO₂NH—, cyano, C₁-C₆ alkyl-CO—NH—, C₁-C₆ alkyl-OOCNH—, C₁-C₆ alkyl-O—, C₁-C₆ alkyl-SO—, C₁-C₆ alkyl-S—, C₁-C₆ alkyl-SO₂—, C₁-C₆ alkyl-C═O—, NH₂—, C₁-C₆ alkyl-NH—, (C₁-C₆ alkyl)₂N—, ArCONH—, ArNHSO₂—, (Ar)₂—N—SO₂—, C₁-C₆ alkyl-NHSO₂—, ArS—, ArSO—, ArSO₂—, ArC═O—, NH₂CONH—C₁-C₆ alkyl-NHCONH—, (C₁-C₆ alkyl)₂-NCONH—, ArNHCONH—, (C₁-C₆)₂—N—SO₂—, Ar—O—, Ar—NH—, Ar(C₁-C₆)N—, or (C₁-C₆)₂NSO₂—; wherein Ar represents phenyl, pyridyl, thienyl or furanyl, optionally substituted with one or more substituents selected from halogen, C₁-C₆ alkyl, C₁-C₆ alkoxy, CF₃, OH, CN, nitro, amino, C₁-C₆ alkyl-NH—, or (C₁-C₆ alkyl)₂N—;

R⁵ and R⁶ may together with the nitrogen atom to which they are attached, form a saturated or unsaturated ring optionally containing one or more further heteroatoms;

X is

(a) NH, or

(b) O, and

Y is S, SO, S₂, O, NH, C═N, or N═C.

Particularly preferred compounds according to the invention are those of Formula I or a pharmaceutically acceptable salt thereof, wherein Het is

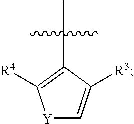

R¹ is CH₃ or CH₂OH;

R² is CH₃, or CH₂CH₃;

R³, and R⁴, are independently hydrogen or C₁-C₆ alkyl;

R⁵ and R⁶ are independently

(a) H,

(b) C₁-C₆ alkyl,

(c) mono or dihydroxylated C₁-C₆ alkyl, or

(d) C₁-C₆ alkoxy-substituted C₁-C₆ alkyl;

X is NH, and

Y is S, O, NH, C═N, or N═C.

Another embodiment of the present invention is a compound of Formula I wherein

Het is

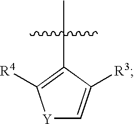

R¹ is H, CH₃, or CH₂OH;

R² is CH₃, or CH₂CH₃;

R³ is C₁-C₆ alkyl;

R⁴ is C₁-C₆ alkyl;

R⁵ and R⁶ are each independently selected from hydrogen, C₁-C₆ alkyl, mono or dihydroxylated C₁-C₆ alkyl, C₁-C₆ alkoxy-(C₁-C₆ alkyl), hydroxylated C₁-C₆ alkoxy-(C₁-C₆ alkyl) or R⁵ and R⁶ may together with the nitrogen atom to which they are attached, form morpholine or hydroxylated pyrrolidine;

X is NH; and

Y is S or O.

Yet another embodiment of the present invention is a compound of Formula I wherein

Het is

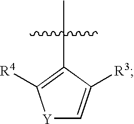

R¹ is CH₃;

R² is CH₃;

R³ is C₁-C₆ alkyl;

R⁴ is C₁-C₆ alkyl;

R⁵ and R⁶ are each independently selected from hydrogen, C₁-C₆ alkyl, mono or dihydroxylated C₁-C₆ alkyl, C₁-C₆ alkoxy-(C₁-C₆ alkyl), hydroxylated C₁-C₆ alkoxy-(C₁-C₆ alkyl) or R⁵ and R⁶ may together with the nitrogen atom to which they are attached, form morpholine or hydroxylated pyrrolidine;

X is NH; and

Y is S or O.

Most preferred compounds of the present invention are

2,3-dimethyl-8-[(2,4-dimethyl-thiophen-3-ylmethyl)-amino]-imidazo[1,2-a]pyridine-6-carboxamide or a pharmaceutically acceptable salt thereof.

Preparation

The present invention also provides the following processes for the manufacture of compounds with the general Formula I.

Process A

Compounds of Formula II

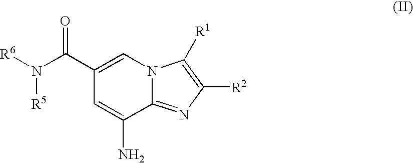

wherein R¹, R², R⁵ and R⁶ are as defined for Formula I can be reacted with compounds of the general Formula III

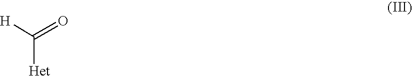

wherein Het is defined for Formula I, in the presence of a Lewis acid, e.g. zinc chloride to compounds of the general Formula IV,

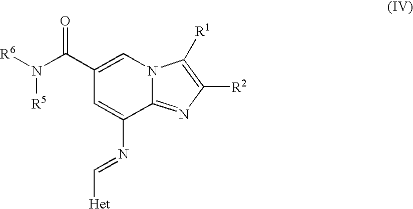

wherein R¹, R², R⁵, R⁶ and Het are as defined for Formula I, whereupon the compounds of the general Formula IV are reduced e.g. by using sodium borohydride or sodium cyanoborohydride to compounds of the general Formula I wherein X is NH. The reactions can be carried out under standard conditions in an inert solvent e.g. methanol or ethanol.

Process B

Compounds of the Formula V,

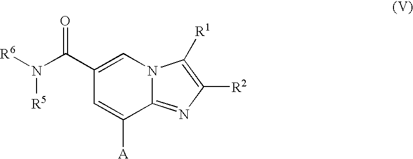

wherein R¹, R², R⁵, R⁶ are as defined for Formula I and A is NH₂ or OH, can be reacted with compounds of the Formula VI

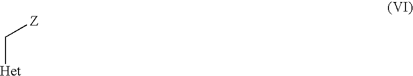

wherein Het is as defined for Formula I and Z is a leaving group, such as a halide, tosyl or mesyl, to the compounds of the Formula I. It is convenient to conduct this reaction in an inert solvent, e.g. acetone, acetonitrile, dimethoxyethane, methanol, ethanol or dimethylformamide with or without a base. The base is e.g. an alkali metal hydroxide, such as sodium hydroxide and potassium hydroxide, an alkali metal carbonate, such as potassium carbonate and sodium carbonate; or an organic amine, such as triethylamine.

Process C

A process for manufacture of compounds with the general Formula I comprises the following steps:

a) Compounds of Formula VII

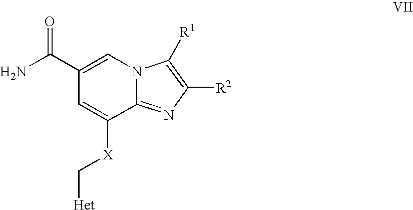

wherein R¹, R², Het and X are as defined in Formula I, can be hydrolyzed under standard conditions to the corresponding carboxylic acid of Formula VIII

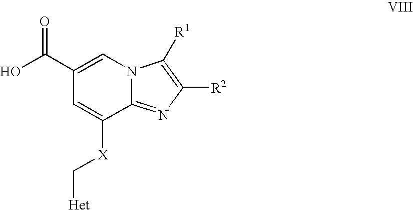

b) Compounds of the Formula VIII wherein R¹, R², Het and X is as defined in Formula I can be reacted with amino compounds of Formula IX

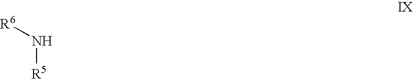

wherein R⁵ and R⁶ are as defined for Formula I, in the presence of a coupling reagent, such as o-Benzotriazol-1-yl-N,N,N′,N′-Tetramethyluronium tetrafluoroborate (TBTU) to the corresponding amide compounds of the Formula I. The reaction can be carried out in an inert solvent under standard conditions.

Medical Use

In a further aspect, the invention relates to compounds of the formula I for use in therapy, in particular for use against gastrointestinal inflammatory diseases. The invention also provides the use of a compound of the formula I in the manufacture of a medicament for the inhibition of gastric acid secretion, or for the treatment of gastrointestinal inflammatory diseases.

The compounds according to the invention may thus be used for prevention and treatment of gastrointestinal inflammatory diseases, and gastric acid-related diseases in mammals including man, such as gastritis, gastric ulcer, duodenal ulcer, reflux esophagitis and Zollinger-Ellison syndrome. Furthermore, the compounds may be used for treatment of other gastrointestinal disorders where gastric antisecretory effect is desirable, e.g. in patients with gastrinomas, and in patients with acute upper gastrointestinal bleeding. They may also be used in patients in intensive care situations, and pre-and postoperatively to prevent acid aspiration and stress ulceration.

The typical daily dose of the active substance varies within a wide range and will depend on various factors such as for example the individual requirement of each patient, the route of administration and the disease. In general, oral and parenteral dosages will be in the range of 5 to 1000 mg per day of active substance.

Pharmaceutical Formulations

In yet a further aspect, the invention relates to pharmaceutical compositions containing at least one compound of the invention, or a therapeutically acceptable salt thereof, as active ingredient.

The compounds of the invention can also be used in formulations together with other active ingredients, e.g. antibiotics such as amoxicillin.

For clinical use, the compounds of the invention are formulated into pharmaceutical formulations for oral, rectal, parenteral or other mode of administration. The pharmaceutical formulation contains a compound of the invention in combination with one or more pharmaceutically acceptable ingredients. The carrier may be in the form of a solid, semi-solid or liquid diluent, or a capsule. These pharmaceutical preparations are a further object of the invention. Usually the amount of active compounds is between 0.1-95% by weight of the preparation, preferably between 0.1-20% by weight in preparations for parenteral use and preferably between 0.1 and 50% by weight in preparations for oral administration.

In the preparation of pharmaceutical formulations containing a compound of the present invention in the form of dosage units for oral administration the compound selected may be mixed with solid, powdered ingredients, such as lactose, saccharose, sorbitol, mannitol, starch, amylopectin, cellulose derivatives, gelatin, or another suitable ingredient, as well as with disintegrating agents and lubricating agents such as magnesium stearate, calcium stearate, sodium stearyl fumarate and polyethylene glycol waxes. The mixture is then processed into granules or pressed into tablets.

Soft gelatin capsules may be prepared with capsules containing a mixture of the active compound or compounds of the invention, vegetable oil, fat, or other suitable vehicle for soft gelatin capsules. Hard gelatin capsules may contain granules of the active compound. Hard gelatin capsules may also contain the active compound in combination with solid powdered ingredients such as lactose, saccharose, sorbitol, mannitol, potato starch, cornstarch, amylopectin, cellulose derivatives or gelatin.

Dosage units for rectal administration may be prepared (i) in the form of suppositories which contain the active substance mixed with a neutral fat base; (ii) in the form of a gelatin rectal capsule which contains the active substance in a mixture with a vegetable oil, paraffin oil or other suitable vehicle for gelatin rectal capsules; (iii) in the form of a ready-made micro enema; or (iv) in the form of a dry micro enema formulation to be reconstituted in a suitable solvent just prior to administration.

Liquid preparations for oral administration may be prepared in the form of syrups or suspensions, e.g. solutions or suspensions containing from 0.1% to 20% by weight of the active ingredient and the remainder consisting of sugar or sugar alcohols and a mixture of ethanol, water, glycerol, propylene glycol and polyethylene glycol. If desired, such liquid preparations may contain coloring agents, flavoring agents, saccharine and carboxymethyl cellulose or other thickening agent. Liquid preparations for oral administration may also be prepared in the form of a dry powder to be reconstituted with a suitable solvent prior to use.

Solutions for parenteral administration may be prepared as a solution of a compound of the invention in a pharmaceutically acceptable solvent, preferably in a concentration from 0.1% to 10% by weight. These solutions may also contain stabilizing ingredients and/or buffering ingredients and are dispensed into unit doses in the form of ampoules or vials. Solutions for parenteral administration may also be prepared as a dry preparation to by reconstituted with a suitable solvent extemporaneously before use.

The compounds according to the invention can also be used in formulations together with other active ingredients, e.g. for the treatment or prophylaxis of conditions involving infection by Helicobacter pylori of human gastric mucosa. Such other active ingredients may be antimicrobial agents, in particular:

β-lactam antibiotics such as amoxicillin, ampicillin, cephalothin, cefaclor or cefixime;

macrolides such as erythromycin, or clarithromycin;

tetracyclines such as tetracycline or doxycycline;

aminoglycosides such as gentamycin, kanamycin or amikacin;

quinolones such as norfloxacin, ciprofloxacin or enoxacin;

others such as metronidazole, nitrofurantoin or chloramphenicol; or

preparations containing bismuth salts such as bismuth subcitrate, bismuth subsalicylate, bismuth subcarbonate, bismuth subnitrate or bismuth subgallate.

The compounds according to the invention can also be used in formulations together with other active ingredients, e.g. for the treatment or prophylaxis of conditions involving medicament induced gastric ulcer. Such other active ingredients may be an NSAID, an NO-NSAID, a COX-2 inhibitor or a bisphosphonate.

Intermediates

A further aspect of the invention is new intermediate compounds which are useful in the synthesis of compounds according to the invention.

Thus, the invention includes compound of Formula (IV)

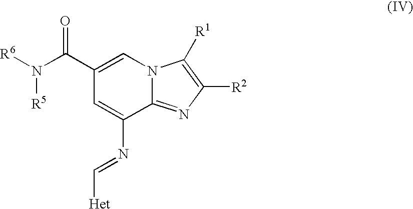

wherein R¹, R², R⁵, R⁶ and Het are as defined for Formula I above.

### EXAMPLES

### 1. PREPARATION OF COMPOUNDS OF THE INVENTION

### Example 1.1

### Synthesis of 8-{[(2,4-dimethylthien-3-yl)methyl]amino)}-2,3-dimethylimidazo[1,2-a]pyridine-6-carboxamide

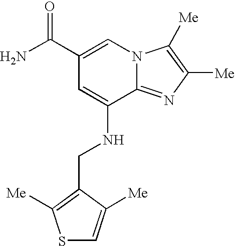

8-Amino-2,3-dimethylimidazo[1,2-a]pyridine-6-carboxamide mesylate (0.36 g, 1 mmol), 2,4-dimethylthiophene-3-carbaldehyde (0.17 g, 1.2 mmol), zinc chloride (0.15 g, 1.1 mmol) and sodium cyanoborohydride (0.14 g, 2.1 mmol) were added to methanol (20 ml) and the mixture was refluxed for 20 h. under nitrogen atmosphere. After cooling to room temperature triethylamine (0.5 ml) was added to the reaction mixture and the solvent was evaporated under reduced pressure. Purification of the residue by column chrormatography on silica gel using methylene chloride:methanol (10:1) as eluent gave 6 mg (2%) of the title compound.

¹H—NMR (300 MHz, DMSO-d₆) 2.15 (s, 3H), 2.25 (s, 3H), 2.35 (s, 3H), 2.45 (s, 3H), 4.3 (d, 2H), 5.4 (t, 1H), 6.6 (s, 1H), 6.9 (s, 1H), 7.3 (bs, 1H), 7.95 (bs, 1H), 8.1 (s, 1H)

### Examples 1.2-1.23

Examples 1.2-1.23 is prepared according to the procedure depicted in Scheme 1, see below.

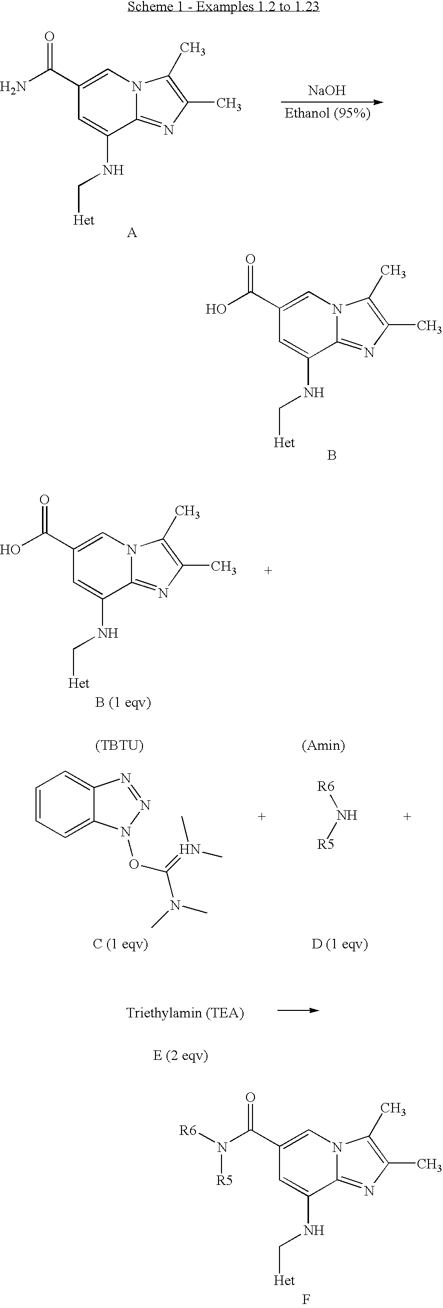

As the starting compound A in the reactions the following compounds were used.

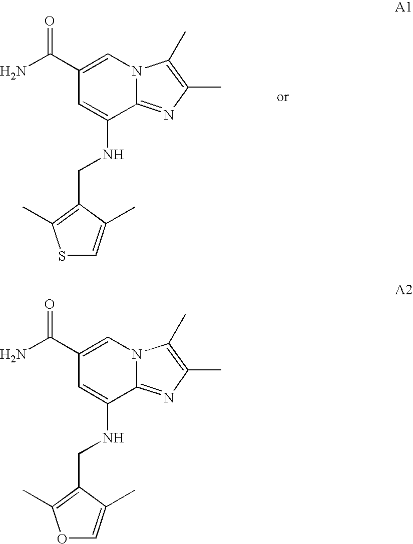

As the reacting compound D the following compounds were used.

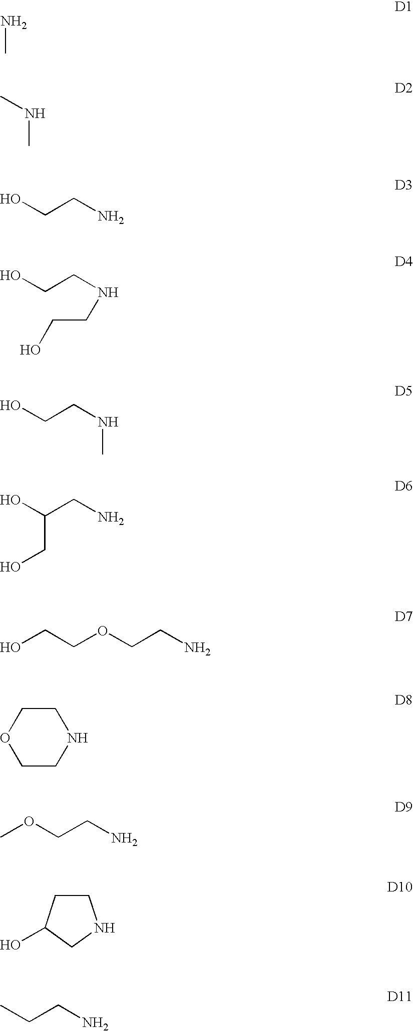

The following compounds F is thus prepared.

| | A1 | A2 |
|---|---|---|
| D1 | Example 1.2 | Example 1.3 |
| D2 | Example 1.4 | Example 1.5 |
| D3 | Example 1.6 | Example 1.7 |
| D4 | Example 1.8 | Example 1.9 |
| D5 | Example 1.10 | Example 1.11 |
| D6 | Example 1.12 | Example 1.13 |
| D7 | Example 1.14 | Example 1.15 |
| D8 | Example 1.16 | Example 1.17 |
| D9 | Example 1.18 | Example 1.19 |
| D10 | Example 1.20 | Example 1.21 |
| D11 | Example 1.22 | Example 1.23 |

General procedure: A (1 eqv) and sodium hydroxide (5 eqv) is dissolved in ethanol (95%) and the mixture is refluxed for 24 h. The solvent is evaporated under reduced pressure and water is added to the residue. The pH is adjusted by addition of acetic acid and the solid that precipitated is isolated by filtration, washed with water and dried to give the title desired compound (B)

B (1 eqv), C (1 eqv), D (1 eqv) and E (2 eqv) are added to dimethylformamide and the mixture is stirred at r.t. for 24 h. The solvent is evaporated under reduced pressure and the residue is purified by chromatography using silica gel and methylene chloride/methanol as eluent to give the desired compound F

TABLE 1
Compounds according to Example 1.2-1.23

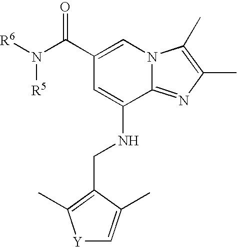

| Ex. # | 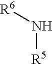 | Y |
|---|---|---|
| 1.2 | 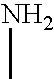 | S |
| 1.3 | 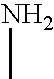 | O |
| 1.4 | 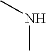 | S |
| 1.5 | 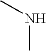 | O |
| 1.6 | 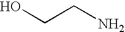 | S |
| 1.7 | 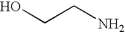 | O |
| 1.8 | 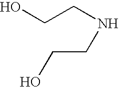 | S |
| 1.9 | 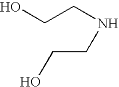 | O |
| 1.10 | 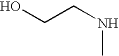 | S |
| 1.11 | 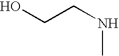 | O |
| 1.12 | 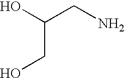 | S |
| 1.13 | 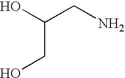 | O |
| 1.14 | 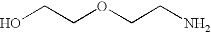 | S |
| 1.15 | 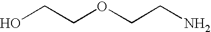 | O |
| 1.16 | 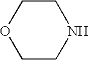 | S |
| 1.17 | 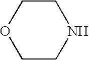 | O |
| 1.18 | 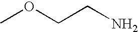 | S |
| 1.19 | 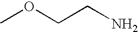 | O |
| 1.20 | 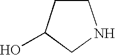 | S |
| 1.21 | 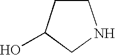 | O |
| 1.22 | 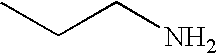 | S |
| 1.23 | 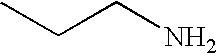 | O |

### BIOLOGICAL TESTS

1. In vitro Experiments

Acid Secretion Inhibition in Isolated Rabbit Gastric Glands

Inhibiting effect on acid secretion in vitro in isolated rabbit gastric glands was measured as described by Berglindh et al. (1976) Acta Physiol. Scand. 97, 401-414.

Determination of H⁺,K⁺-ATPase Activity

Membrane vesicles (2.5 to 5 μg) were incubated for 15 min at +37° C. in 18 mM Pipes/Tris buffer pH 7.4 containing 2 mM MgCl₂, 10 mM KCl and 2 mM ATP. The ATPase activity was estimated as release of inorganic phosphate from ATP, as described by LeBel et al. (1978) Anal. Biochem. 85, 86-89.

2. In Vivo Experiments

Inhibiting Effect on Acid Secretion in Female Rats

Female rats of the Sprague-Dawly strain are used. They are equipped with cannulated fistulae in the stomach (lumen) and the upper part of the duodenum, for collection of gastric secretions and administration of test substances, respectively. A recovery period of 14 days after surgery is allowed before testing commenced.

Before secretory tests, the animals are deprived of food but not water for 20 h. The stomach is repeatedly washed through the gastric cannula with tap water (+37° C.), and 6 ml Ringer-Glucose given subcutaneously. Acid secretion is stimulated with infusion during 2.5-4 h (1.2 ml/h, subcutaneously) of pentagastrin and carbachol (20 and 110 nmol/kg·h, respectively), during which time gastric secretions are collected in 30-min fractions. Test substances or vehicle are given either at 60 min after starting the stimulation (intravenous and intraduodenal dosing, 1 ml/kg), or 2 h before starting the stimulation (oral dosing, 5 ml/kg, gastric cannula closed). The time interval between dosing and stimulation may be increased in order to study the duration of action. Gastric juice samples are titrated to pH 7.0 with NaOH, 0.1 M, and acid output calculated as the product of titrant volume and concentration.

Further calculations are based on group mean responses from 4-6 rats. In the case of administration during stimulation; the acid output during the periods after administration of test substance or vehicle are expressed as fractional responses, setting the acid output in the 30-min period preceding administration to 1.0. Percentage inhibition is calculated from the fractional responses elicited by test compound and vehicle. In the case of administration before stimulation; percentage inhibition is calculated directly from acid output recorded after test compound and vehicle.

Bioavailability in Rat

Adult rats of the Sprague-Dawley strain are used. One to three days prior to the experiments all rats are prepared by cannulation of the left carotid artery under anaesthesia. The rats used for intravenous experiments are also cannulated in the jugular vein (Popovic (1960) J. Appl. Physiol. 15, 727-728). The cannulas are exteriorized at the nape of the neck.

Blood samples (0.1-0.4 g) are drawn repeatedly from the carotid artery at intervals up to 5.5 hours after given dose. The samples are frozen until analysis of the test compound.

Bioavailability is assessed by calculating the quotient between the area under blood/plasma concentration (AUC) curve following (i) intraduodenal (i.d.) or oral (p.o.) administration and (ii) intravenous (i.v.) administration from the rat or the dog, respectively.

The area under the blood concentration vs. time curve, AUC, is determined by the log/linear trapezoidal rule and extrapolated to infinity by dividing the last determined blood concentration by the elimination rate constant in the terminal phase. The systemic bioavailability (F %) following intraduodenal or oral administration is calculated as F(%)=(AUC(p.o. or i.d.)/AUC(i.v.))×100.

Inhibition of Gastric Acid Secretion and Bioavailability in the Conscious Dog.

Labrador retriever or Harrier dogs of either sex are used. They are equipped with a duodenal fistula for the administration of test compounds or vehicle and a cannulated gastric fistula or a Heidenhaim-pouch for the collection of gastric secretion.

Before secretory tests the animals are fasted for about 18 h but water is freely allowed. Gastric acid secretion is stimulated for up to 6.5 h infusion of histamine dihydrochloride (12 ml/h) at a dose producing about 80% of the individual maximal secretory response, and gastric juice collected in consecutive 30-min fractions. Test substance or vehicle is given orally, i.d. or i.v., 1 or 1.5 h after starting the histamine infusion, in a volume of 0.5 ml/kg body weight. In the case of oral administration, it should be pointed out that the test compound is administered to the acid secreting main stomach of the Heidenham-pouch dog.

The acidity of the gastric juice samples is determined by titration to pH 7.0, and the acid output calculated. The acid output in the collection periods after administration of test substance or vehicle are expressed as fractional responses, setting the acid output in the fraction preceding administration to 1.0. Percentage inhibition is calculated from fractional responses elicited by test compound and vehicle.

Blood samples for the analysis of test compound concentration in plasma are taken at intervals up to 4 h after dosing. Plasma is separated and frozen within 30 min after collection and later analyzed. The systemic bioavailability (F %) after oral or i.d. administration is calculated as described above in the rat model.

1. A compound of the formula I 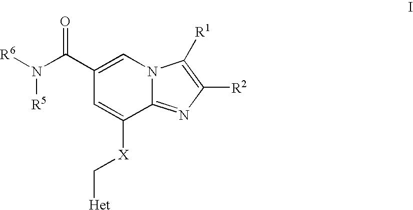
or a pharmaceutically acceptable salt thereof, wherein
Het is a 4-, 5-, or 6-membered aromatic or aliphatic heterocyclic group containing at least one nitrogen, oxygen or sulphur atom, substituted with a R³ and a R⁴ group in the ortho positions;
R¹ is H, CH₃, or CH₂OH;
R² is CH₃ or CH₂ CH₃
R³, and R⁴ are independently selected from the group of H, C₁-C₆ alkyl, hydroxylated C₁-C₆ alkyl, or halogen;
R⁵ and R⁶ are independently selected substituents, comprising C, H, N, O, S, Se, P and halogen atoms, which give compounds of Formula I a molecular weight≦600 and;
X is NH, or O.

2. A compound or salt thereof according to claim 1, wherein
Het is 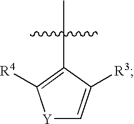
R¹ is H, CH₃, or CH₂OH;
R² is CH₃ or CH₂CH₃;
R³ and R⁴ are independently hydrogen, C₁-C₆ alkyl, hydroxylated C₁-C₆ alkyl or halogen;
R⁵ and R⁶ are independently selected substituents, comprising C, H, N, O, S, Se, P and halogen atoms, which give compounds of Formula I a molecular weight≦600;
X is NH or O; and
Y is S, SO, SO₂, O, NH, C═N, or N═C.

3. A compound or salt thereof according to claim 1, wherein
Het is 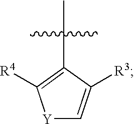
R¹ is CH₃ or CH₂OH;
R² is CH₃ or CH₂CH₃;
R³, and R⁴ are independently H, C₁-C₆ alkyl,hydroxylated C₁-C₆ alkyl, or halogen;
R⁵ and R⁶ are independently
(a) H,
(b) C₁-C₆ alkyl,
(c) hydroxylated C₁-C₆ alkyl,
(d) C₁-C₆ alkoxy-substituted C₁-C₆ alkyl,
(e) C₂-C₆ alkenyl,
(f) C₂-C₆ alkynyl,
(g) halogenated C₁-C₆ alkyl,
(h) C₃-C₈ cycloalkyl,
(i) cycloalkyl-substituted C₁-C₆ alkyl,
(j) aryl, in which aryl represents phenyl, pyridyl, thienyl or furanyl, optionally substituted by one or more substituents selected from halogen, C₁-C₆ alkyl, C₁-C₆ alkoxy, CF₃, OH, nitro, amino, C₁-C₆ alkyl-NH—, (C₁-C₆ alkyl)₂-N—, or CN,
(k) aryl substituted C₁-C₆ alkyl, in which aryl represents phenyl, pyridyl, thienyl or furanyl, optionally substituted with one or more substituents selected from halogen, C₁-C₆ alkyl, C₁-C₆ alkoxy, CF₃, OH, nitro, amino C₁-C₆ alkyl-NH—, (C₁-C₆ alkyl)₂-N—, or CN,
(l) R⁸-alkyl, in which R⁸ is NH₂C═O—, C₁-C₆ alkyl-NHC═O—, (C₁-C₆ alkyl)₂NC═O—, C₁-C₆ alkyl-OOC—, NH₂SO₂—, C₁-C₆ alkyl-SO₂NH—, ArSO₂NH—, cyano, C₁-C₆ alkyl-CO—NH—, C₁-C₆ alkyl-OOCNH—, C₁-C₆ alkyl-O—, C₁-C₆ alkyl-SO—, C₁-C₆ alkyl-S—, C₁-C₆ alkyl-SO₂—, C₁-C₆ alkyl-C═O—, NH₂—, C₁-C₆ alkyl-NH—, (C₁-C₆ alkyl)₂N—, ArCONH—, ArNHSO₂—, (Ar)₂—N—SO₂—, C₁-C₆ alkyl-NHSO₂—, ArS—, ArSO—, ArSO₂—, ArC═O—, NH₂CONH—C₁-C₆ alkyl-NHCONH—, (C₁-C₆ alkyl)₂-NCONH—, ArNHCONH—, (C₁-C₆)₂—N—SO₂—, Ar—O—, Ar—NH—, Ar(C₁-C₆)N—, or (C₁-C₆)₂NSO₂—; wherein Ar represents phenyl, pyridyl, thienyl or furanyl, optionally substituted with one or more substituents selected from halogen, C₁-C₆ alkyl, C₁-C₆ alkoxy, CF₃, OH, CN, nitro, amino, C₁-C₆ alkyl-NH—, or (C₁-C₆ alkyl)₂N—;
X is NH, or O; and
Y is S, SO, SO₂, O, NH, C═N, or N═C.

4. A compound or salt thereof according to claim 1, wherein
Het is 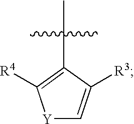
R¹ is CH₃ or CH₂OH;
R² is CH₃, orCH₂CH₃;
R³, and R⁴, are independently hydrogen and C₁-C₆ alkyl;
R⁵ and R⁶ are independently H, C₁-C₆ alkyl, hydroxylated C₁-C₆ alkyl, or C₁-C₆ alkoxy-substituted C₁-C₆ alkyl;
X is NH, and
Y is S, O, NH, C═N, or N═C.

5. A compound according to claim 1, wherein
Het is 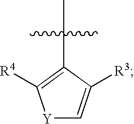
R¹ is H, CH₃, or CH₂OH;
R² is CH₃, or CH₂CH₃;
R³ is C₁-C₆ alkyl;
R⁴ is C₁-C₆ alkyl;
R⁵ and R⁶ are each independently selected from hydrogen, C₁-C₆ alkyl, hydroxylated C₁-C₆ alkyl, C₁-C₆ alkoxy-(C₁-C₆ alkyl), hydroxylated C₁-C₆ alkoxy-(C₁-C₆ alkyl) or R⁵ and R⁶ may together with the nitrogen atom to which they are attached, form morpholine or hydroxylated pyrrolidine;
X is NH; and
Y is S or O.

6. A compound according to claim 1, wherein
Het is 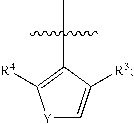
R¹ is CH₃;
R² is CH₃;
R³ is C₁-C₆ alkyl;
R⁴ is C₁-C₆ alkyl;
R⁵ and R⁶ are each independently selected from hydrogen, C₁-C₆ alkyl, hydroxylated C₁-C₆ alkyl, C₁-C₆ alkoxy-(C₁-C₆ alkyl), hydroxylated C₁-C₆ alkoxy-(C₁-C₆ alkyl) or R⁵ and R⁶ may together with the nitrogen atom to which they are attached, form morpholine or hydroxylated pyrrolidine;
X is NH; and
Y is S or O.

7. The compound or salt thereof according to claim 1 being 8-{[(2,4-dimethylthien-3-yl)methyl]amino}-2,3-dimethylimidazo[1,2-a]pyridine-6-carboxamide.

8. A process for the preparation of a compound according to any one of claims 1 to 7, comprising the steps of reacting a compound of Formula II 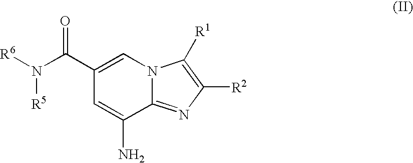
wherein R¹, R², R⁵ and R⁶ are as defined in claim 1 with compounds of the Formula III 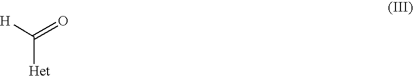
wherein Het is defined in claim 1, in the presence of a Lewis acid, e.g. zinc chloride to compounds of the Formula IV, 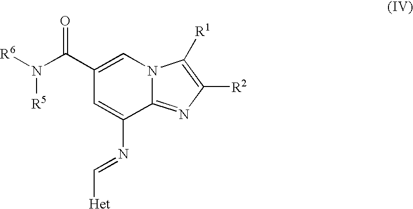
which is thereafter reduced under standard conditions, e.g. by using sodium borhydride or sodiumcyano borhydride in an inert solvent such as methanol or ethano, to give compounds of Formula I as defined in claim 1 wherein X is NH.

9. A process for the preparation of a compound according to any one of claims 1 to 7, comprising the steps of reacting a compound of Formula V, 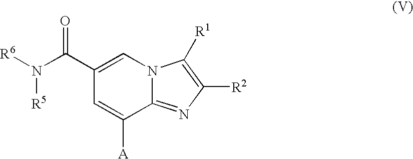
wherein R¹, R², R⁵, R⁶ are as defined in claim 1 and A is NH₂ or OH, with compounds of the Formula VI 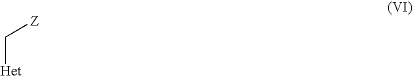
wherein Het is as defined claim 1 and Z is a leaving group, such as a halide, tosyl or mesyl, in an inert solvent, such as acetone, acetonitrile, dimethoxyethane, methanol, ethanol or dimethylformamide with or without a base such as an alkali metal hydroxide, or an organic amine, to give the compounds of the Formula I as defined in claim 1.

10. A process for the preparation of a compound according to any one of claims 1 to 7, comprising the steps of
a)hydrolyzing a compound of Formula VII 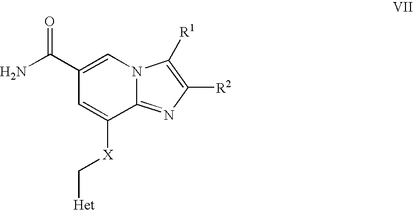
wherein R¹, R², Het and X are as defined in Formula I, under standard conditions to the corresponding carboxylic acid of Formula VIII 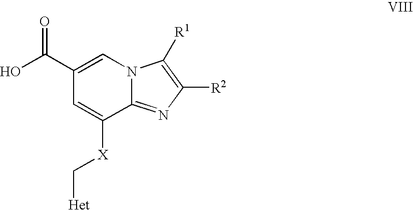
b) Reacting a compound of the Formula VIII wherein R¹, R², Het and X is as defined in Formula I with an amino compound of Formula IX 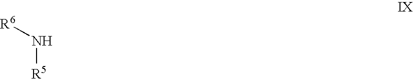
wherein R⁵ and R⁶ are as defined for Formula I, in the presence of a coupling reagent, such as o-Benzotriazol-1-yl-N,N,N′,N′-Tetramethyluronium tetrafluoroborate (TBTU), in an inert solvent under standard conditions, to give the corresponding amide compound of the Formula I. The reaction can be carried out.

11. A compound according to any one of claims 1 to 7 for use in therapy as an acid pump inhibiting drug.

12. A pharmaceutical formulation containing a compound according to any one of claims 1 to 7 as active ingredient in combination with a pharmaceutically acceptable diluent or carrier.

13. Use of a compound according to any one of claims 1 to 7 for the manufacture of a medicament for the inhibition of gastric acid secretion.

14. Use of a compound according to any one of claims 1 to 7 for the manufacture of a medicament for the treatment of gastrointestinal inflammatory diseases.

15. Use of a compound according to any one of claims 1 to 7 the manufacture of a medicament for the treatment or prophylaxis of conditions involving infection by Helicobacter pylori of human gastric mucosa, wherein the said compound is adapted to be administered in combination with at least one antimicrobial agent.

16. A method for inhibiting gastric acid secretion which comprises administering to a mammal, including man, in need of such inhibition an effective amount of a compound according to any one of claims 1 to 7.

17. A method for the treatment of gastrointestinal inflammatory diseases which comprises administering to a mammal, including man, in need of such treatment an effective amount of a compound according to any one of claims 1 to 7.

18. A method for the treatment or prophylaxis of conditions involving infection by Helicobacter pylori of human gastric mucosa, which comprises administering to a mammal, including humans, in need of such treatment an effective amount of a compound as claimed in any one of claims 1 to 7, wherein the said salt is administered in combination with at least one antimicrobial agent.

19. A compound of the formula (IV) 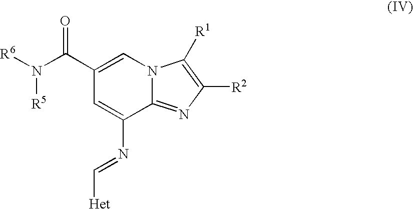
wherein R¹, R², R⁵, R⁶ and Het are as defined in claim 1.
